(19) United States
(12) Patent Application Publication
(10) Pub. No.: US 2003/0036709 A1
(43) Pub. Date: Feb. 20, 2003

(54) Combined anesthesia and biopsy guide

(76) Inventor: George Lee Jordan III, Madison Heights, MI (US)

Correspondence Address: B. Delano Jordan, 1524 Ingram Terrace, Silver Spring, MD, 20906, US

(21) Appl. No.: 09/928,400

(22) Filed: Aug. 14, 2001

(51) Int. Cl. A61B 10/00

(52) U.S. Cl. 600/562

(57) ABSTRACT

A biopsy guide enables biopsies to be performed more quickly and with less patient discomfort. The biopsy guide includes a body portion having surfaces defining an ultrasound aperture for receiving an ultrasound transducer. A biopsy fitting is moveably coupled to the body portion, where the biopsy fitting has surfaces defining a biopsy aperture for receiving a biopsy device. The body portion has a syringe guide for aligning an anesthetic syringe with a trajectory of the biopsy device.

### FIELD OF THE INVENTION

1. Technical Field

The present invention generally relates to ultrasound guided biopsies. More particularly, the invention relates to a biopsy guide and method for conducting a biopsy that enables the alignment of an anesthetic syringe with a trajectory of a biopsy device.

2. Discussion

Biopsies involve the removal of a small piece of living tissue from an organ or other part of the body for microscopic examination and have become an integral part of modern medicine. For example, the procedure can be useful in establishing diagnoses, estimating prognoses, and following the course of a disease. In fact, biopsies are commonly used to treat the liver, lymph node, pancreas, and any other mass in the abdomen, pelvis, or chest.

A number of devices and systems have been developed over the years to assist practitioners with biopsies. In particular, ultrasound guided biopsy systems have been useful in conducting percutaneous biopsies. Under this approach, an ultrasound transducer is coupled to a biopsy guide in order to properly locate the subject tissue or tumor. A conventional biopsy guide 60 is shown in FIG. 5 and has a body portion 62 in addition to a biopsy fitting 64, where the body portion 62 has surfaces 66 defining an ultrasound aperture 68. The ultrasound aperture 68 receives the ultrasound transducer (not shown), while a lateral member 70 extends away from the ultrasound aperture 68. The lateral member 70 typically has a “hook” shape for receiving the biopsy fitting 64. The biopsy fitting (or clip) 64 typically has one or more biopsy apertures 72 designed to accommodate various sized biopsy devices. It is the biopsy device that extracts the tissue sample after being guided through the biopsy aperture 72 and along the desired trajectory.

While the above-described biopsy guide 60 has been used for a number of years, certain difficulties remain. For example, in order to reduce patient discomfort and speed up the biopsy procedure, deep local anesthesia is often required. The conventional biopsy guide 60, however, fails to provide an adequate mechanism for anesthetizing the biopsy tract. Thus, the practitioner often must hold the biopsy guide/ultrasound assembly in one hand and administer the anesthesia with a syringe being held freely in the other hand. Furthermore, the practitioner must estimate the subsequent trajectory of the biopsy device when administering the anesthesia. Thus, in addition to the relatively cumbersome aspect of this approach, it is particularly difficult to anesthetize the biopsy tract with the desired level of accuracy. The result can therefore be discomfort to the patient due to insufficient anesthetization of the correct region. It is therefore desirable to provide a system and method for conducting a biopsy that enables the practitioner to confidently and routinely anesthetize the tract of the biopsy device.

The above and other objectives are provided by a biopsy guide in accordance with the principles of the present invention. The biopsy guide includes a body portion having surfaces defining an ultrasound aperture for receiving an ultrasound transducer. A biopsy fitting is moveably coupled to the body portion, where the biopsy fitting has surfaces defining a biopsy aperture for receiving a biopsy device. The body portion has a syringe guide for aligning an anesthetic syringe with a trajectory of the biopsy device. Thus, by incorporating the syringe guide into the biopsy guide, more accurate and reliable anesthetization can be achieved.

Further in accordance with the present invention, a syringe guide for a biopsy guide is provided. The syringe guide has surfaces defining a syringe aperture for receiving an anesthetic syringe. The syringe guide further includes surfaces defining a fitting aperture for receiving a biopsy fitting. A hinged section couples the syringe guide to the biopsy fitting such that a central axis of the biopsy fitting can be rotatably aligned with a central axis of the syringe aperture.

In another aspect of the invention, a method for conducting a biopsy includes the step of imaging tissue with an ultrasound transducer, wherein the ultrasound transducer is coupled to a biopsy guide. A tract is anesthetized through a syringe aperture of the biopsy guide based on a signal from the ultrasound transducer. The method further provides for extracting a portion of the tissue through a biopsy aperture of the biopsy guide, where the biopsy aperture and the syringe aperture are co-axial.

It is to be understood that both the foregoing general description and the following detailed description are merely exemplary of the invention, and are intended to provide an overview or framework for understanding the nature and character of the invention as it is claimed. The accompanying drawings are included to provide a further understanding of the invention, and are incorporated in and constitute part of this specification. The drawings illustrate various features and embodiments of the invention, and together with the description serve to explain the principles and operation of the invention.

### BRIEF DESCRIPTION OF THE DRAWINGS

The various advantages of the present invention will become apparent to one skilled in the art by reading the following specification and subjoined claims and by referencing the following drawings, in which:

FIG. 1A is a side view of a biopsy guide in accordance with the principles of the present invention;

FIG. 1B is an enlarged view of the syringe guide shown in FIG. 1A;

FIG. 2 is a top view of the biopsy guide shown in FIG. 1;

FIG. 3 is an illustration of a conventional ultrasound image, useful in understanding the invention;

FIG. 4A is a side view of a biopsy fitting removable gauge insert in accordance with the principles of the present invention;

FIG. 4B is a top view of the removable gauge insert shown in FIG. 4a; and

FIG. 5 is a top view of a conventional biopsy guide, useful in understanding the invention.

### DETAILED DESCRIPTION OF THE PREFERRED EMBODIMENTS

The following description of the preferred embodiment(s) is merely exemplary in nature and is in no way intended to limit the invention, its application, or uses.

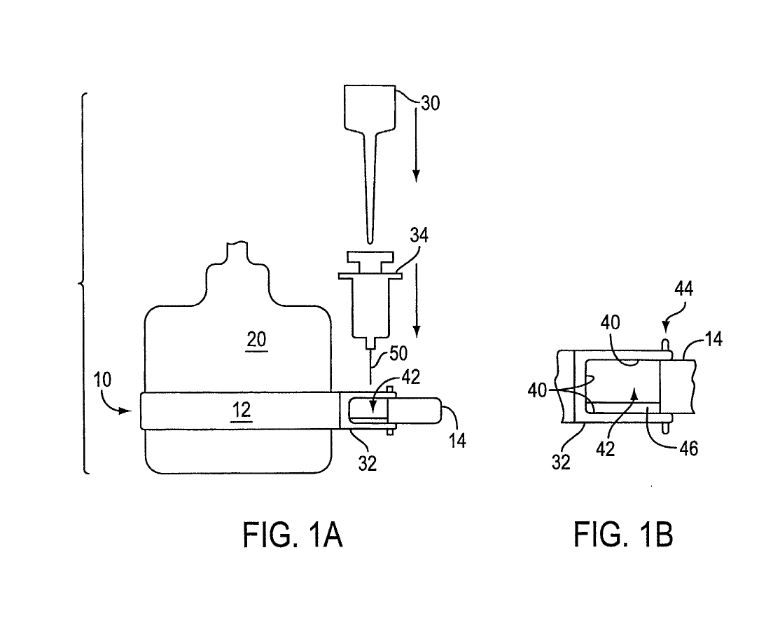

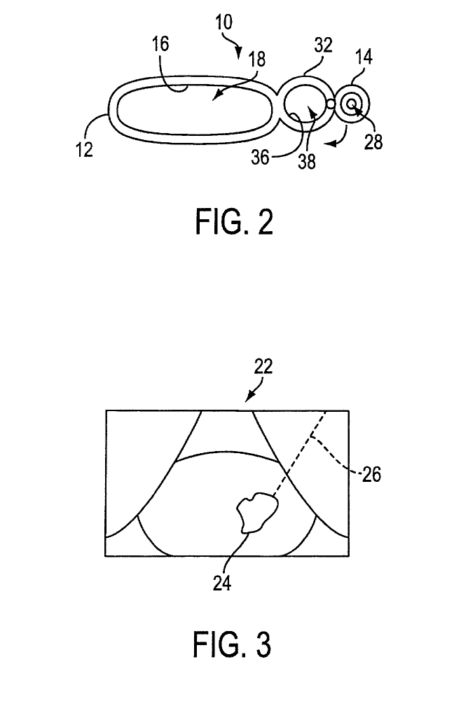

Turning now to FIG. 1A, a biopsy guide in accordance with the principles of the present invention is shown generally at 10. The biopsy guide 10 can be used for ultrasound guided percutaneous biopsies and provides a number of significant advantages. With continuing reference to FIGS. 1A and 2, it can be seen that the biopsy guide 10 generally includes a body portion 12 and a biopsy fitting 14. The body portion 12 has surfaces 16 defining an ultrasound aperture 18 for receiving an ultrasound transducer 20. The ultrasound transducer 20 is capable of generating ultrasound images such as the image 22 shown in FIG. 3. Thus, the ultrasound transducer enables tissue such as tumor 24 to be located and a biopsy trajectory (or tract) 26 to be defined. The trajectory 26 is the path that a biopsy device 30 takes in order to sample the tumor 24.

Returning now to FIGS. 1A and 2, it can be seen that the biopsy fitting 14 is moveably coupled to the body portion 12, where the biopsy fitting 14 has surfaces defining a biopsy aperture 28 for receiving the biopsy device 30. It is important to note, however, that local anesthesia is typically required before extracting the tissue sample. It should also be noted that conventional ultrasound guided biopsy systems fail to provide a mechanism for accurately anesthetizing the intended trajectory 26 (FIG. 3) of the biopsy device 30. In response to this shortcoming, the body portion 12 is equipped with a syringe guide 32 for aligning an anesthetic syringe 34 with the trajectory of the biopsy device 30.

Specifically, it is preferred that the syringe guide 32 has surfaces 36 defining a syringe aperture 38 for receiving the anesthetic syringe 34. As best seen in FIG. 1B, the syringe guide 32 further includes surfaces 40 defining a fitting aperture 42 for receiving the biopsy fitting 14. A hinged section 44 couples the syringe guide 32 to the biopsy fitting 14 such that a central axis of the biopsy fitting 14 can be rotatably aligned with a central axis of the syringe aperture 38.

It can also be seen that the syringe guide 32 includes a stop member 46 for ensuring that the two axes are properly aligned. In fact, the stop member 46 preferably has a stabilizing mechanism such as a magnetic coupling to prevent unwanted movement once the axes are aligned. In this regard, either the stop member 46, the biopsy fitting 14, or both can be made of a metallic material and/or magnetically polarized material. Other approaches could involve adhesives, hook mechanisms, or other appropriate fixing devices.

It can therefore be seen that the syringe aperture 38 preferably has an inner diameter that is approximately equal to an outer diameter of the anesthetic syringe 34. An example inner diameter might be designed to match a 10 cubic centimeter syringe (having a needle 50) as is commonly used in biopsy procedures. It is preferred that the inner diameter of the syringe aperture 38 is adjustable so as to maximize the adaptability of the biopsy guide 10 to various procedures. It can also be seen that the syringe aperture 38 extends longitudinally through the syringe guide 32, whereas the fitting aperture 42 extends laterally through the syringe guide 32. This design enables the biopsy guide 10 to have a relatively simple construction wherein space is optimized.

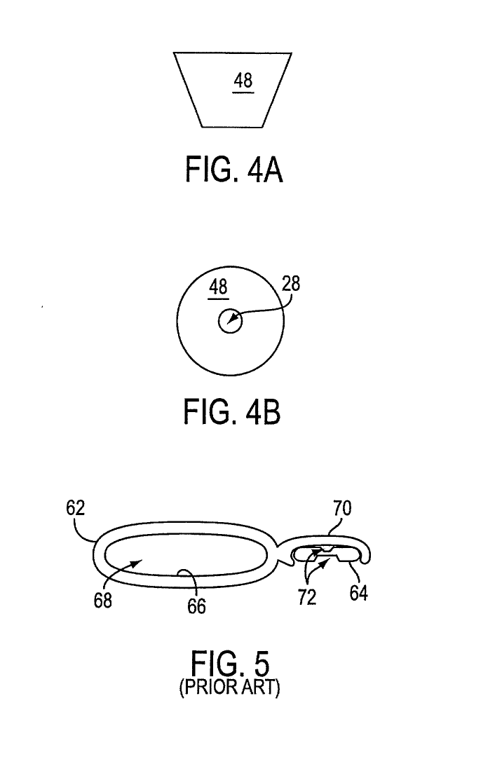

Turning now to FIGS. 2, 4A, and 4B, it can be seen that the biopsy fitting 14 preferably includes a removable gauge insert 48 such that the biopsy aperture 28 is adjustable. It is envisioned that the gauge insert 48 can have an inner diameter between 10 and 20 gauge, depending upon the size of the biopsy device 30 being used. Although the insert 48 is shown as being a drop-in type of insert, other approaches can also be used. For example, the insert 48 can be equipped with threads that mate with threads of the biopsy fitting 14 to enable the two parts to be screwed together.

Thus, with continuing reference to FIGS. 1A-4B, it can be seen that the biopsy guide 10 enables biopsies to be conducted in a unique and more effective manner. For example, the tissue 24 can be imaged with the ultrasound transducer 20, wherein the ultrasound transducer 20 is coupled to the biopsy guide 10. The tract (or trajectory) 26 is then anesthetized through a syringe aperture 38 of the biopsy guide 10 based on the signal from the ultrasound transducer 20. The invention then provides for extracting a portion of the tissue 24 through a biopsy aperture 28 of the biopsy guide 10, where the biopsy aperture 28 and the syringe aperture 38 are co-axial. Specifically, by rotating the biopsy fitting 14 into the laterally extending fitting aperture 42 after anesthetizing the tract 26, the two apertures 28, 38 can be made to be co-axial. Furthermore, by selecting the appropriately sized removable gauge insert 48 and positioning the removable gauge insert 48 within the biopsy fitting 14, the biopsy aperture 28 can be made to be effectively adjustable.

Thus, the present invention enables biopsies to be performed more quickly and with less patient discomfort. The biopsy guide 10 can be constructed of various materials such as metal and/or plastic, depending upon cost, reliability, and maintenance concerns.

Those skilled in the art can now appreciate from the foregoing description that the broad teachings of the present invention can be implemented in a variety of forms. Therefore, while this invention can be described in connection with particular examples thereof, the true scope of the invention should not be so limited since other modifications will become apparent to the skilled practitioner upon an study of the drawings, specification and following claims.

What is claimed is:

1. A biopsy guide comprising:
a body portion having surfaces defining an ultrasound aperture for receiving an ultrasound transducer; and
a biopsy fitting movably coupled to the body portion, the biopsy fitting having surfaces defining a biopsy aperture for receiving a biopsy device;
said body portion having a syringe guide for aligning an anesthetic syringe with a trajectory of the biopsy device.

2. The biopsy guide of claim 1 wherein the syringe guide includes:
surfaces defining a syringe aperture for receiving the anesthetic syringe;
surfaces defining a fitting aperture for receiving the biopsy fitting; and
a hinged section coupling the syringe guide to the biopsy fitting such that a central axis of the biopsy fitting can be rotatably aligned with a central axis of the syringe aperture.

3. The biopsy guide of claim 2 wherein the syringe aperture has an inner diameter approximately equal to an outer diameter of the anesthetic syringe.

4. The biopsy guide of claim 3 wherein the inner diameter is approximately equal to an outer diameter of a ten cubic centimeter syringe.

5. The biopsy guide of claim 2 wherein the syringe aperture extends longitudinally through the syringe guide.

6. The biopsy guide of claim 2 wherein the fitting aperture extends laterally through the syringe guide.

7. The biopsy guide of claim 1 including a metal material.

8. The biopsy guide of claim 1 including a plastic material.

9. The biopsy guide of claim 1 wherein the biopsy fitting includes a removable gauge insert such that the biopsy aperture is adjustable.

10. The biopsy guide of claim 9 wherein the biopsy aperture has an inner diameter between ten and twenty gauge.

11. A syringe guide for a biopsy guide, the syringe guide comprising:
surfaces defining a syringe aperture for receiving an anesthetic syringe;
surfaces defining a fitting aperture for receiving a biopsy fitting; and
a hinged section coupling the syringe guide to the biopsy fitting such that a central axis of the biopsy fitting can be rotatably aligned with a central axis of the syringe aperture.

12. The syringe guide of claim 11 wherein the syringe aperture has an inner diameter approximately equal to an outer diameter of the anesthetic syringe.

13. The syringe guide of claim 12 wherein the inner diameter is approximately equal to an outer diameter of a ten cubic centimeter syringe.

14. The syringe guide of claim 11 wherein the syringe aperture extends longitudinally through the syringe guide.

15. The syringe guide of claim 11 wherein the fitting aperture extends laterally through the syringe guide.

16. A biopsy guide for percutaneous ultrasound guided biopsies, the biopsy guide comprising:
a body portion having surfaces defining an ultrasound aperture for receiving an ultrasound transducer; and
a biopsy fitting movably coupled to the body portion, the biopsy fitting having surfaces defining a biopsy aperture for receiving a biopsy device, the biopsy fitting further including a removable gauge insert such that the biopsy aperture is adjustable;
said body portion having a syringe guide for aligning an anesthetic syringe with a trajectory of the biopsy device;
said syringe guide having surfaces defining a syringe aperture extending longitudinally through the syringe guide for receiving the anesthetic syringe;
said syringe guide further including surfaces defining a fitting aperture extending laterally through the syringe guide for receiving the biopsy fitting;
said syringe guide further including a hinged section coupling the syringe guide to the biopsy fitting such that an axis of the biopsy fitting can be rotatably aligned with an axis of the syringe aperture.

17. The biopsy guide of claim 16 wherein the biopsy aperture has an inner diameter between ten and twenty gauge.

18. A method for conducting a biopsy, the method comprising the steps of:
imaging tissue with an ultrasound transducer wherein the ultrasound transducer is coupled to a biopsy guide;
anesthetizing a tract through a syringe aperture of the biopsy guide based on a signal from the ultrasound transducer; and
extracting a portion of the tissue through a biopsy aperture of the biopsy guide, the biopsy aperture and the syringe aperture being co-axial.

19. The method of claim 18 further including the step of rotating a biopsy fitting containing the biopsy aperture into a laterally extending fitting aperture of the syringe guide after anesthetizing the tract.

20. The method of claim 19 further including the step of positioning a removable gauge insert within the biopsy fitting such that the biopsy aperture is adjustable.
